(19) United States
(12) Patent Application Publication
(10) Pub. No.: US 2011/0084230 A1
(43) Pub. Date: Apr. 14, 2011

(54) Process for Synthesizing Organoelemental Compounds

(76) Inventors: Paul Knochel, Gauling (DE); Andrei Gavryushin, Germering (DE); Vladimir Malakhov, Muenchen (DE); Arkady Krasovskiy, Singapore (SG)

(21) Appl. No.: 12/225,647

(22) PCT Filed: Apr. 3, 2007
(86) PCT No.: PCT/EP2007/053229

§ 371 Date: Nov. 19, 2010

(30) Foreign Application Priority Data
Apr. 3, 2006 (DE) 10 2006 015 378.2

(51) Int. Cl. C07F 3/06; C07C 69/76; C07C 255/50; C07C 49/303; C07C 251/00; C07C 15/02; C07D 307/02; C07D 307/34; C07C 1/32; C09K 3/00

(52) U.S. Cl. 252/182.3; 556/121; 556/33; 560/51; 558/411; 568/332; 564/271; 585/24; 549/483; 568/657; 585/436

(57) ABSTRACT

The present application discloses a process for preparing a compound of the general formula R¹-M¹-A_{d}.zLiX (I) by reacting a compound R¹-A (III) with an element M¹ in the presence of lithium salts. The application also discloses a process for preparing a compound of the general formula R¹ₘ-M³-Tₘ.zLiX (II) by reacting a compound R¹-A (III) with an M³-containing compound in the presence of lithium salts and in the presence of an elemental metal M². The metal M³ may be selected from Al, Mn, Cu, Zn, Sn, Ti, In, La, Ce, Nd, Y, Li, Sm, Bi, Mg, B, Si and S.

The present invention refers to the preparation of organo element compounds starting from organo halogen compounds, to the organo element compounds themselves as well as to the use of these organo element compounds.

Hereinafter, the basic principle of the invention shall be explained by use of organo zinc compounds. However, the invention shall not be limited to organo zinc compounds but can be carried out with a lot of other metals or semimetals (metalloids).

Due to their specific reactivity and tolerance for many functional groups, organo zinc compounds are important starting or intermediate products in organic chemistry. The direct preparation of, for example, organo zinc bromides directly from aryl and alkyl bromides has, however, been largely limited by the use of the comparatively expensive and less stable Rieke zinc or by a reaction procedure in pure dimethylacetamide (DMAC) as solvent hitherto.

For preparing Rieke zinc, zinc chloride is reduced with lithium naphthaline to a fine dispersed zinc powder. Due to its large surface, this zinc powder is highly reactive. It can be inserted in a carbon-halogen bond. Due to its high reactivity, it can, however, also react with other functional groups that are present in a molecule and can thus cause undesired side reactions and by-products. Hitherto, an isolation of the organo zinc compounds has not been possible.

The insertion of magnesium in carbon-halogen bonds is known as the Grignard reaction. The solubility of Grignard compounds can be enhanced by adding lithium ions, as it is, for example, disclosed in EP 1 582 524. In EP 1 582 524, there is disclosed a method for replacing an organic moiety at a magnesium ion. Similar methods for the preparation of organo element compounds do not exist for other metals or metalloids.

It is therefore an object of the present invention to provide a simplified method for the synthesis of organo element compounds starting from organo halogen compounds. Furthermore, it is an object of the present invention to provide novel organo element compounds as pure chemical substances or in solution, respectively. Another object of the present invention is to provide methods for reacting the novel organo element compounds as well as the reaction products themselves.

According to the invention, these objects are solved by the features of the independent claims.

As the inventors of the present invention recently found out, a reaction between a metallic element and organo halogen compounds can efficiently be carried out in a solution containing lithium ions. Functional groups as, for example, esters or nitriles, are tolerated in this method. The method is thus applicable to a number of organic compounds which are also able to carry different functional groups.

The present invention discloses a method for preparing a compound having the general formula

R¹-M¹-A_{d}zLiX (I)

by reacting a compound R¹-A (III) with an element M′ in presence of LiX,
wherein
R¹ is a substituted or un-substituted C₃-C₂₄ aryl or C₃-C₂₄ heteroaryl containing one or more heteroatoms like B, O, N, S, Se, P or Si, a linear or branched, substituted or un-substituted C₁-C₂₀ alkyl, C₂-C₂₀ alkenyl or C₂-C₂₀ alkynyl or a substituted or un-substituted C₃-C₂₀ cycloalkyl or a derivative thereof;
M¹ is an element selected from Mn, Cu, Zn, Sn, In, La, Ce, Nd, Y, Li, Sm, Na, K and Bi;
A is a halogen selected from F, Cl, Br, I; a sulphonate (RSO₃—) or a phosphonate (—OP(O)(OR)₂) wherein R is defined like R¹;
d is 0 or 1;
z is >0; and
X is selected from the group consisting of F; Cl; Br; CN; SCN; NCO; Hal¹Oₖ, wherein k=3 or 4 and Hal¹ is selected from Cl, Br and I; NO₃; BF₄; PF₆; H; a carboxylate having the general formula R^{x}CO₂; a disilazide having the general formula (R^{x}₃Si)₂N; a thiolate having the general formula SR^{x}; an alcoholate having the general formula OR^{x}; R^{x}P(O)O₂; or SCOR^{x}; an amine having the general formula R^{x}NH; a dialkyl- or diaryl amine having the general formula R^{x}₂N, wherein R^{x}is defined as below or R^{x}₂N represents a cyclic alkylamine; a phosphine having the general formula PR^{x}₂, wherein R^{x} is defined as below or PR^{x}₂ represents a cyclic phosphine; OⱼSR^{x}, wherein j=2 or 3; or NOᵣ, wherein r=2 or 3; and derivatives thereof; wherein R^{x} is a substituted or un-substituted C₄-C₂₄ aryl or a C₃-C₂₄ heteroaryl containing one or more heteroatoms like B, O, N, S, Se, P or Si; a linear or branched, substituted or un-substituted C₁-C₂₀ alkyl; C₂-C₂₀ alkenyl or C₂-C₂₀ alkynyl; or a substituted or un-substituted C₃-C₂₀ cycloalkyl; or derivatives thereof; or H. Tosylate (p-toluene sulphonate) or mesylate (methane sulphonate) are preferably used as sulphonates.

The present method thereby has the advantage that an element, especially an elementary metal, can be used in any form. The element or metal can, for example, be used in form of granules, swarf, bars, sheets or as a powder. By the addition of a lithium salt, a reaction is facilitated or enabled. A highly fine dispersion as it is, for example the case for Rieke zinc, is not necessary. Any compound having a carbon-halogen bond can be used as the organic starting compound R¹-A (III). The metal is inserted in this carbon-halogen bond according to the method of the present invention. Other functional groups that are present in the molecule are not altered in the method and do not interfere with the reaction according to the invention. Thereby, multiply functionalised molecules can be used in the reaction according to the invention. This grants access to a plurality of differently functionalised molecules having a carbon element halogen group.

According to this aspect of the present invention, the number d is 0 or 1. The value of n thereby conforms to the valence of the element M¹. The valence of the element M¹ thereby corresponds to the valency or the oxidation number. If this valence is set to v, so d=v−1. Hence, for example, the value of d=0, for a monovalent metal M¹ like Li. For a bivalent metal such as Zn, the value of d=1.

According to a second aspect of the invention, a compound having the general formula

R¹ₘ-M³-TₙzLiX (II)

can be obtained by reacting a compound R¹-A (III) with a M³-containing compound in the presence of LiX and in the presence of an elementary metal M². M² is thereby selected from Li, Na, K, Cs, Mg, Ca, Mn and Zn. R¹, z, A and X are as defined above and M³ is defined as M¹ above, wherein M³ can additionally be Al, Ti, Mg, B, Si and S. M³ is also selected from the group consisting of Al, Mn, Cu, Zn, Sn, Ti, In, La, Ce, Nd, Y, Li, Sm, Bi, Mg, B, Si and S. T is defined as A or X above, i.e. T can be selected from A and/or from X, wherein X and T can be identical or different. n is 0, 1, 2 or 3 m is 1, 2 or 3. If m=2 or m=3, there are several moieties R¹ bonded with a single element M³. With respect to the definition of R¹ above, these moieties R¹ can be identical or different moieties.

According to this aspect of the present invention, an insertion and transmetalation reaction is performed in one single step. Thereby, the element M³ of the M³-containing compound is less reactive than the metal M². Thus, the M³ elements which are otherwise not accessible for a direct reaction, can be inserted in the compound (III) under mild conditions. The insertion reaction can be carried out by using a reactive metal M² which can be easily activated. Subsequently, the element M³ in form of a M³-containing compound is inserted into the organic compound by a transmetalation reaction under mild conditions. It is therefore important that the element M³ is less reactive than the element M².

The M³-containing compound can be a salt, specifically a metal salt, an organo element compound, specifically an organo metal compound, or also an organo element salt compound, preferably an organo metal salt compound. As already noted above for M¹ and d, both n and m depend from the valency of the element M³. In this context, the terms valency, valence and oxidation number are equivalently used. For the valence v of M³ with the numbers n and m, the relation v=m+n applies.

According to another aspect of the present invention, there is provided a compound having the general formula R¹ₘ-M³-TₙzLiX (II) wherein R¹, M³, m, n, z, X and T are defined as above, wherein M³ does not comprise Mg.

According to still a further aspect of the present invention, there is provided a solution of a compound having the general formula R¹ₘ-M³-TₙzLiX (II) in a solvent, wherein R¹, M³, m, n, z, X and T are defined as above and wherein M³ does not comprise Mg. Or, in other words, the present invention relates to a composition in form of a solution containing a compound having the formula (II) in a solvent.

According to a further aspect of the present invention, there is provided a reaction of a compound having the general formula R¹ₘ-M³-TₙzLiX (II) with an electrophile, wherein R¹, M³, m, n, z, X and T are defined as above and wherein M³ does not comprise Mg. In principle, there can be used many different types of electrophiles. For example, electrophiles that are mentioned in the following documents but are not limited thereto can be used:
a) Handbook of Grignard reagents; edited by Gary S. Silverman and Philip E. Rakita (Chemical Industries; v. 64).
b) Grignard reagents New Developments; edited by Herman G. Richey, Jr., 2000, John Wiley & Sons Ltd.
c) Methoden der Organischen Chemie, Houben-Weyl, vol. XIII/2a, Metallorganische Verbindungen Be, Mg, Ca, Sr, Ba, Zn Cd. 1973.
d) The chemistry of the metal-carbon bond, vol. 4. edited by Frank R. Hartley. 1987, John Wiley & Sons.

A final aspect of the present invention relates to a product of a reaction of an electrophile with a compound having the general formula R¹ₘ-M³-TₙzLiX (II) wherein R¹, M³, m, n, z, X and T are defined as above, wherein M³ does not comprise Mg. The possible electrophiles can again be selected from the documents mentioned under a) to d) but are not limited thereto. The compounds (II) thereby react as a nucleophile. They can thus be used in reactions, in which nucleophiles can be used.

The solvent for the methods of the present invention as well as for the solution and the reaction according to the present invention can be selected from the group consisting of cyclic, linear or branched mono- or polyethers, thioethers, amines, phosphines and derivatives thereof that contain one or more additional heteroatoms selected from O, N, S and P, preferably tetrahydrofurane (THF), 2-methyltetrahydrofurane, dibutylether, diethylether, tert-butylmethylether, dimethoxyethane, dioxanes, preferably 1,4-dioxane, triethylamine, ethyldiisopropylamine, dimethylsulphide, dibutylsulphide; cyclic and linear amides, preferably N-methyl-2-pyrrolidone (NMP), N-ethyl-2-pyrrolidone (NEP), N-butyl-2-pyrrolidone (NBP), N,N-dimethylformamide (DMF), N,N-dimethylacetamide (DMAC); cyclic, linear or branched alkanes and/or alkenes wherein one or more hydrogen atoms are replaced by halogens, preferably dichloromethane, 1,2-dichloroethane, CCl₄; derivates of urea, preferably N,N′-dimethylpropylene urea (DMPU), N,N,N′N′-tetramethyl urea; aromatic, heteroaromatic or aliphatic hydrocarbons, preferably, benzene, toluene, xylene, pyridine, pentane, cyclohexane, hexane, heptane; hexamethylphosphortriamide (HMPA), CS₂; or combinations thereof.

The presence of lithium ions in the solution for preparing the compound having the general formula (I) or in the solution itself, enables the reaction or the dissolution of the compound, respectively. Thereby, a lithium salt can be used stoichiometrically in relation to the organo halogen compound (III), wherein z=1. However, it is also possible to only use traces of lithium salt. Then z is >0. On the other hand, it is also possible to introduce the lithium salt excessively when compared with the organo halogen compound, wherein z is then greater than 1. Within all aspects of the present invention, z is preferably within the range from 0.01 to 5, preferably from 0.5 to 2, more preferably from 0.9 to 1.2, and most preferably about 1.

The M³-containing compounds being used according to the second aspect of the present invention are compounds which can contain a metal, a metalloid or a non-metal M³, for example, in a salt, a covalent bond or a complex. Thereby, metal-halogen compounds, metal-alkyl-, metal-aryl-, metal-alkoxy or metal-aryloxy compounds are preferably used. More preferably used compounds that contain M³ are MgBr₂, MgCl₂, B(OMe)₃, B(iPrO)₃, BF₃, Et₂AlCl, Si(OMe)₄, SiCl₄, MnCl₂, SnCl₂, ZnCl₂, ZnBr₂, TiCl(OiPr)₃, Ti(OiPr)₄, InCl₃, LaCl₃, CeCl₃, SmCl₃ and NdCl₃. Thereby, Me represents methyl and iPr iso-propyl.

The concentration of lithium chloride in the solution of the present invention is from 0.01 to 5 mol/l, preferably from 0.1 to 4 mol/l. A concentration of from 0.2 to 1.5 mol/l is most preferred. The concentration of the M³-containing compound is preferably from 1 to 4 mol/l, more preferably 1.2 to 3 mol/l and most preferably about 1.4 mol/1.

The elementary metals being used in this reaction can be activated by known compounds. Thereby, there can be used all compounds known to activate elementary metals for a reaction. The elements M¹ and M² can, for example, be activated by compounds selected from the group consisting of copper salts such as, for example, CuCl₂, CuBr₂ or CuSO₄, nickel salts such as, for example, NiCl₂ or NiSO₄, iron compounds such as, for example, FeCl₂ or FeCl₃, cobalt compounds such as, for example, CoCl₂ or CoSO₄, I₂, C₂H₄Br₂, Cl(CH₂)₂Br, t-BuOLi, BCl₃, BF₃, LiBH₄, LiAlH₄, NaAlH₄, Et₃Al, DIBAL-H (diisobutylaluminum hydride), Na[H₂Al(OCH₂CH₂OCH₃], Me₃SiCl, Et₂Zn, ICl and SnCl₂. For example, magnesium swarf can be activated with 2 to 3 mol % Me₃SiCl. The reaction procedure can be carried out at room temperature.

When, in the context of the present invention, a metal is mentioned, those metalloids or non-metals that are accessible for the reaction such as, for example, boron, silicon or sulphur are also encompassed. The metals Zn, Mn, La, Ce, Nd and Sm are preferred for M¹, wherein zinc is specifically preferred. In the selection of M², Li, Mg and Na are preferred metals. Zn, B, Si and Sn are preferred elements in the selection of M³.

The terms alkyl, alkenyl and alkynyl relate to linear, cyclic and branched, substituted and un-substituted C₁ or C₂ to C₂₀ compounds, respectively. Preferred ranges for these compounds are C₁ to C₁₀, preferably C₁ to C₅ (lower alkyl), for alkyl or C₂ to C₁₀, preferably C₂ to C₅, for alkenyl or alkynyl, respectively. Linear or branched, substituted or un-substituted C₃ to C₂₀ cycloalkanes are understood as cycloalkyl. A preferred range is C₃ to C₁₅ and more preferably C₃ to C₈.

With aryl are meant substituted or un-substituted C₃ to C₂₄ aryl compounds. Heteroaryls are substituted or un-substituted C₃ to C₂₄ heteroaryl compounds containing one or more heteroatoms like B, O, N, S, Se, P or Si. Preferable ranges for both are C₄ to C₁₅ or even more preferably C₄ to C₁₀.

Whenever any one of the moieties R, R^{x} or R¹ is substituted with a substituent, the substituent can be selected from any substituent known to a person skilled in the art. A person skilled in the art will select a possible substituent in accordance with his expertise and he will be able to select a substituent that will not interact with other substituents that are present in the molecule and that will not interfere with reactions or interact in these reactions, specifically not in reactions being described in this application. Possible substituents include the following, without being limited thereto:
halogens, preferably fluorine, chlorine, bromine and iodine;
aliphatic, alicyclic, aromatic and heteroaromatic hydrocarbons, specifically alkanes, alkenes, alkynes, aryls, arylidenes, heteroaryls and heteroarylidenes;
carboxylic acids including salts and esters thereof;
carboxylic acid halides
aliphatic, alicyclic, aromatic or heteroaromatic carboxylic acid esters;
aldehydes;
aliphatic, alicyclic, aromatic or heteroaromatic ketones;
alcohols and alcoholates including hydroxyl groups;
phenols and phenolates;
aliphatic, alicyclic, aromatic or heteroaromatic ethers;
aliphatic, alicyclic, aromatic or heteroaromatic peroxides;
hydroxy peroxides;
aliphatic, alicyclic, aromatic or heteroaromatic amides or amidines;
nitriles;
aliphatic, alicyclic, aromatic or heteroaromatic amines;
aliphatic, alicyclic, aromatic or heteroaromatic imines;
aliphatic, alicyclic, aromatic or heteroaromatic sulphides, and a thiol group;
sulfonic acids including salts and esters thereof;
thiols and thiolates;
phosphonic acids including salts and esters thereof;
phosphinic acids including salts and esters thereof;
phosphorous acids including salts and esters thereof;
phosphinous acids including salts and esters thereof.

The substitutents can be bonded to the moieties via a carbon atom, an oxygen atom, a nitrogen atom, a sulphur atom or a phosphorus atom. N, O, S and P are preferably used as heteroatoms in e.g. heteroaromates.

The principle underlying all aspects of the present invention is the preparation or use of organo element compounds in the presence of lithium ions. These lithium ions enable or facilitate the reaction of the elementary metals M¹ and M². Moreover, due to the presence of lithium salts in the reaction solution or the compound according to formula (I), the solubility is enhanced and the further reaction is enabled or facilitated.

The compounds having the general formula (I) all share the general formula (II). The method for preparing the compounds having the general formula (II) shall thereby encompass Mg, B, Si and S for the element M³, wherein Mg shall be excluded in the selection of the elements for M³ for the compound according to formula (II) or for the solution of the compound according to formula (II).

For the preparation of organo element compounds according to the general formula R¹-M¹-A_{d} zLiX (I) according to the invention, an organo compound R¹-A is reacted in a solvent with an element, specifically a metal, in the presence of a lithium salt. Thereby, the metal can be used stoichiometrically in relation to the organo compound or preferably excessively. The reaction can be carried out within a temperature range of from −90° C. to 100° C., preferably from 0° C. to 80° C. and most preferably between 15° C. and 60° C. Preferably, a reaction is carried out in an inert gas atmosphere. As the inert gas, for example, nitrogen or Argon can, be used.

In the reaction with elementary metals, the organo element compound according to formula (I) or (II) can further be reacted with an electrophile in situ. However, it is also possible to isolate the organo element compound (I) or (II) and thus, to separate it from excessive elementary metal. If excessive metal is not separated in advance of a further reaction with an electrophile, the metal could react with another carbon-halogen bond that is present in the organic compound. By using a corresponding processing, it is thus possible to selectively react one carbon-halogen group or several carbon-halogen groups that are present in an organic compound.

In the compounds having the formula (II), it is possible that n=2. If this is the case, T₂ could be a bivalent anion being selected from the group consisting of diamines, dialkoxides or dithiols. Thereby, the diamine can preferably have the general formula R′NH—R—NHR′, the dialkoxide can have the general formula HO—R—OH and the dithiol can have the general formula HS—R—SH, wherein R′ and R are independently selected from the same group as R^{x}, wherein R is a bivalent moiety. The limitation for R shall be applied insofar as that no chemically nonsensical compounds will result. Accordingly, the moiety referred to as an alkyl moiety in the selection of R^{x} is an alkanediyl in the selection of R, the alkenyl is an alkenediyl and the alkynyl is an alkynediyl. A preferred diamine is CH₃NHCH₂CH₂NHCH₃ and preferred dialkoxides are the dialkoxides of the dioles HOCH₂CH₂OH, binole and 1,2-diaminocyclohexane.

If there are several anions T present in the compound (II), these can be both identical or different. For example, one anion can be derived from the use of a compound (III) and another anion can be derived from the M³-containing compound. Thus, the anions T can be independently selected from each other.

The reaction of organo halogen compounds with a metal M² in the presence of a lithium salt and a M³-containing compound in situ enables an easy access to compounds (II) with metals M³ that are otherwise only preparable under harder conditions. Thus, an easy access to compounds (II) is enabled which are otherwise only available under more difficult conditions.

With the methods of the present invention, accesses to organo element compounds (II) are provided which have previously not been accessible.

In the following, the reaction of the invention shall be illustrated by use of general examples, however, without being limited to these examples.

It is, for example, possible, to react metallic zinc with alkyl bromides in THF in the presence of LiCl at 50° C. to the corresponding alkyl zinc bromides with a high yield. A general work instruction includes heating an alkyl bromide in a 0.7 M (saturated at room temperature) solution of lithium chloride in THF with three equivalents of zinc powder. Zinc powder is thereby activated with 2 mol % CH₂Br₂ and 2-5 mol % Me₃SiCl. The reaction is carried out at 50° C. in 2-48 hours. The alkyl zinc bromides obtained thereby can be scavenged with different electrophiles. Additionally, there can be used catalysts such as, for example, palladium for accelerating the reaction. The structures and the yield of some products which can be synthesised in this way are summarised in scheme 1 below.

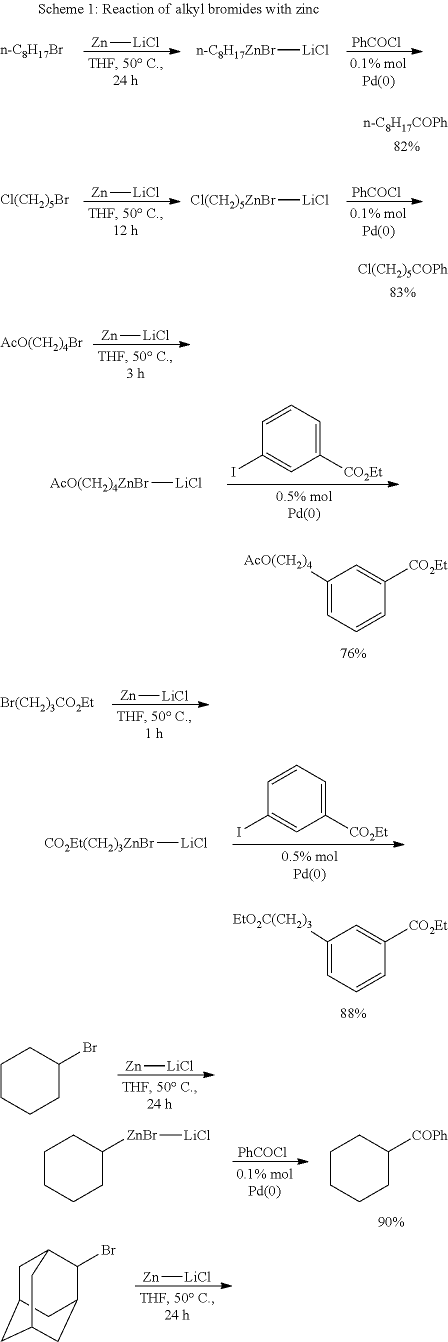

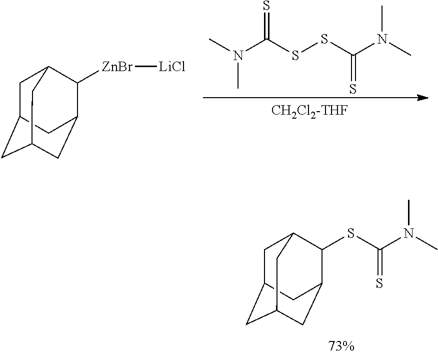

It is also possible to use aryl iodides as starting compounds. Thereby, zinc is inserted in the aryl-iodine bond in the presence of LiCl. A selection of compounds that can be synthesised in accordance with the present invention is given in scheme 2. Subsequently, the zinc organic compounds are reacted with an electrophile. This reaction is carried out quantitatively or mostly approximately quantitatively.

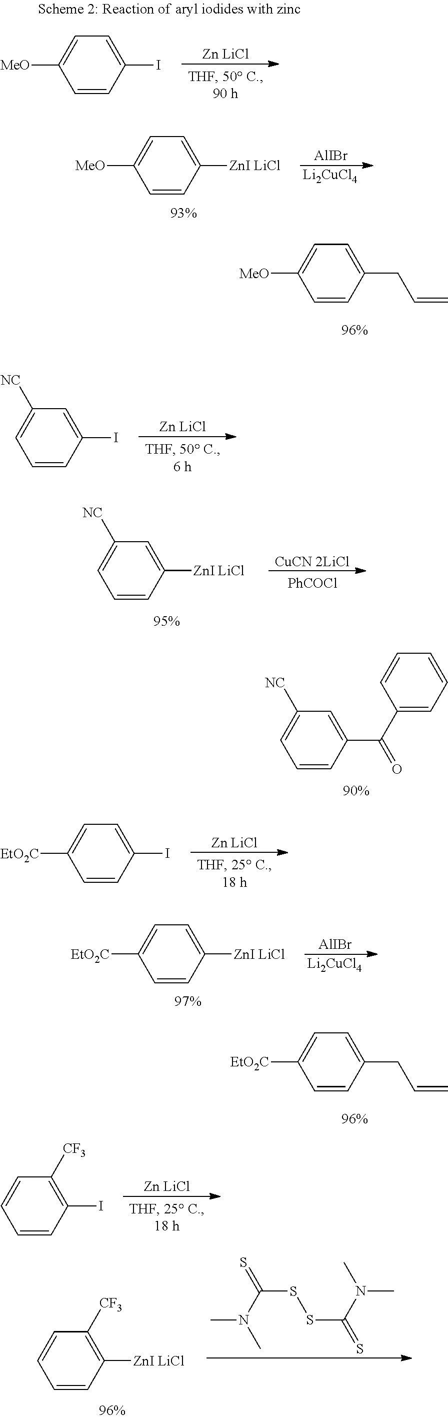

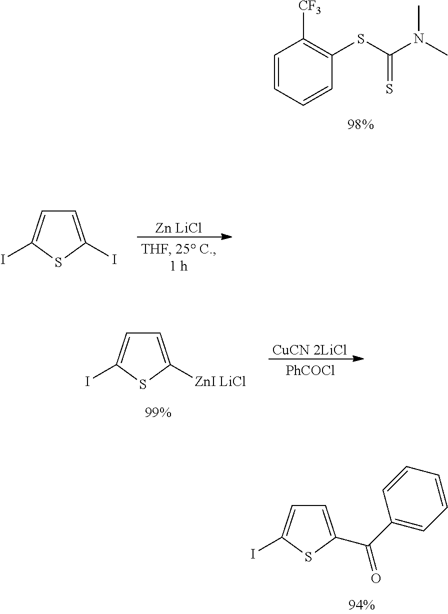

Furthermore, it is possible to prepare the compounds of the present invention starting from metal-containing compounds such as metal-containing salts or organo metal compounds. So, for example, aryl or alkyl bromides can be directly reacted with metallic magnesium and ZnCl₂ in THF in the presence of lithium chloride to aryl or alkyl zinc compounds. The concentration of lithium chloride in the solution is thereby from 1 to 5 mol/1, preferably from 2 to 4 mol/l. A concentration of 2.2 mol/l is especially preferred. The concentration of the M³ containing compound is preferably 1 to 4 mol/l, more preferably 1.2 to 3 mol/l, and most preferably about 1.4 mol/l. The metals used can be activated. For example, magnesium swarf can be activated with 2 to 3 mol % Me₃SiCl. The reaction procedure can be carried out at room temperature. A summary of possible reactions is given in scheme 3. Here, the intermediate zinc organic compounds are again reacted with an electrophile. Thereby, the electrophile can again be a halogen whereby a re-halogenation can result as illustrated in the second example in scheme 3.

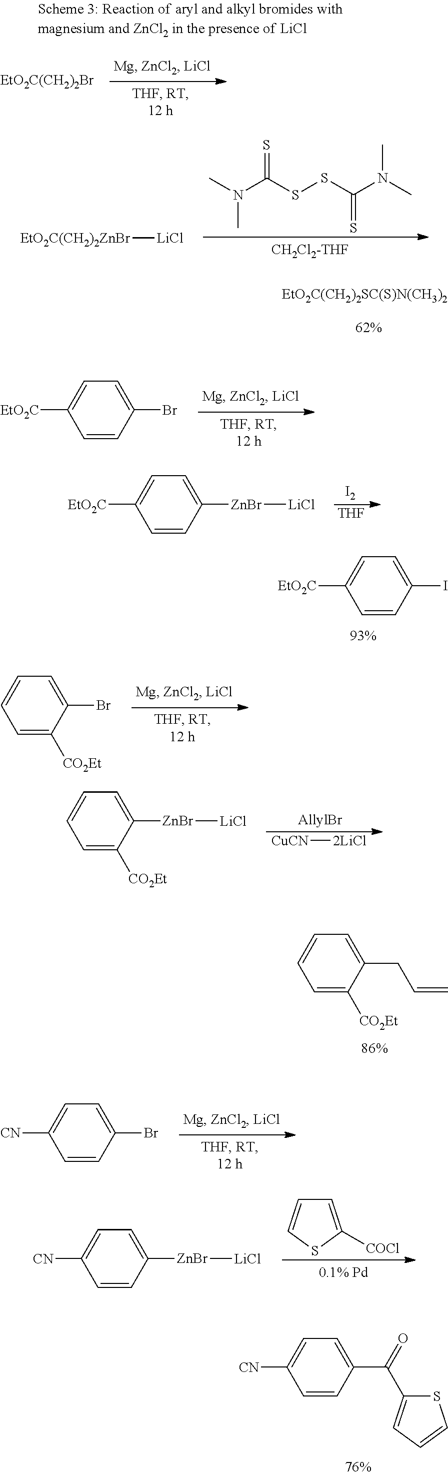

According to another embodiment of the present invention, it is possible to prepare organo element compounds in the presence of LiCl starting from organo halogen compounds and to scavenge these compounds with an electrophile in situ. For example, 4-chloro-benzotrifluoride reacts with lithium in THF in the presence of naphthalene (15 mol %), LiCl and boron acid trimethylester to 4-trifluoromethylphenyl boron acid (see scheme 4). The post-processing of the product is at first carried out in basic medium, then in acid medium, wherein the yield is 42%.

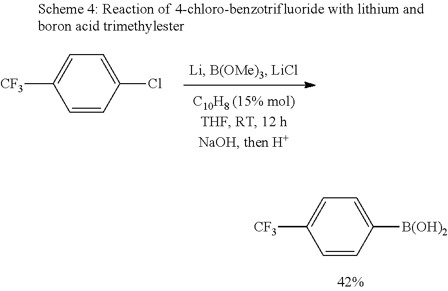

A reaction of 4-chloro-benzotrifluoride with magnesium in THF in the presence of LiCl and Et₂AlCl yields 72% of the corresponding aryl aluminum compound which can then be scavenged with iodine or another electrophile in situ such as illustrated in scheme 5.

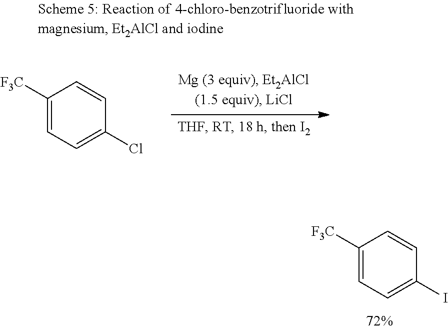

Manganese can also be inserted in a halogen-carbon bond. For example, elementary manganese reacts with n-octyl iodide under mild reaction conditions at room temperature in the presence of lithium chloride to the corresponding insertion product as illustrated in scheme 6.

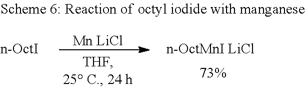

The method illustrated above can analogously be applied to the metals Cu, Bi, Al and In.

The reaction of multiply halogenated organic compounds can be selectively carried out at one or all carbon-halogen bonds. A selective insertion of zinc into a single carbon-iodine bond can, for example, be carried out by using zinc, as illustrated in the following scheme 7. The subsequent transmetalation with a copper species and the reaction with allyl bromide (AllBr) results in the single allylated product with high yield.

2,5-diiodothiophene can be reacted to the mono-substituted product with an excessive of zinc and by subsequently decanting for separating the solution from the remaining zinc. The second substitution of iodine of the thiophene can then result in a thiophene that is differently substituted in the 2- and 5-positions in a further reaction with zinc. However, if the zinc is not decanted or filtered, i.e. removed from the reaction mixture, after the first reaction, the carbonyl group will also be attacked by the alkyl bromide. Thus, the bi-allylated product results.

If, starting with 2,5-diiodothiophene, the solution of zinc is not decanted or filtered in the subsequent reaction, i.e. the zinc is present in the reaction mixture during the whole reaction procedure, the thiophene will be directly bi-substituted.

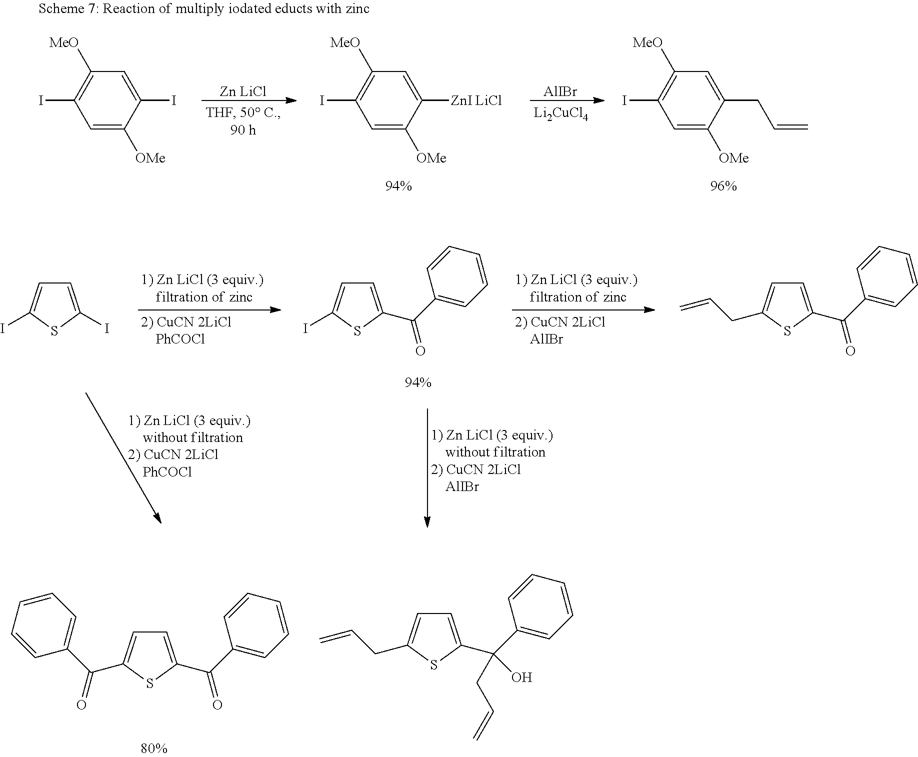

It is also possible to insert zinc in carbon-halogen bonds of aza heterocycles such as, for example, pyridine, quinoline and isoquinoline. The corresponding reactions can be carried out at room temperature and result in, for example, 24 hours in the desired organo zinc compounds with yields of more than 95%. Exemplary compounds obtainable in this way are presented in scheme 8.

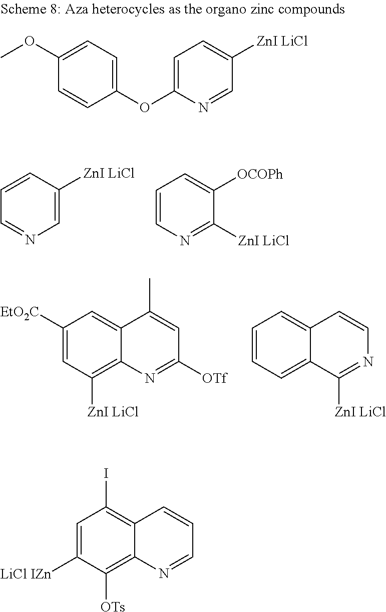

The new method according to the invention can also be used for the synthesis of alkenyl zinc compounds. In the case of Z-iodooctene, the corresponding octenyl zinc iodide has been obtained with a yield of more than 80%. The following reaction with allyl bromide (AllBr) is carried out after a transmetalation with copper with a yield of 72% as illustrated in the upper chemical equation in scheme 9. There, the ratio of the Z- to the E-isomer is 3 to 1.

An insertion of cyclopropyl derivatives in carbon-halogen bonds can also be carried out in accordance with the present invention. While a partial inversion of the configuration can be observed in both cases illustrated in scheme 9 below, these examples are of large interest as such an insertion has been carried out in those systems for the first time. Analogously to the example of iodooctene given above, the reaction of the organo zinc compound with allyl bromide is carried out after a transmetalation with copper with a yield of 75% (see scheme 9).

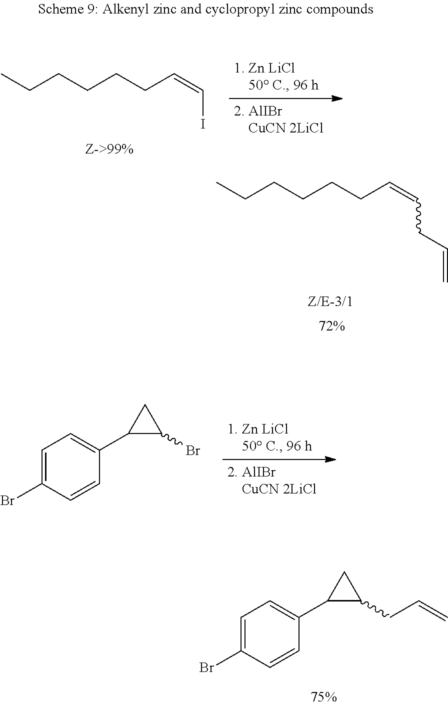

In activated systems, it is also possible to use bromides as starting materials instead of the more expensive iodides. In asymmetrical substrates, a regioselective insertion can be carried out as illustrated in the following example in scheme 10.

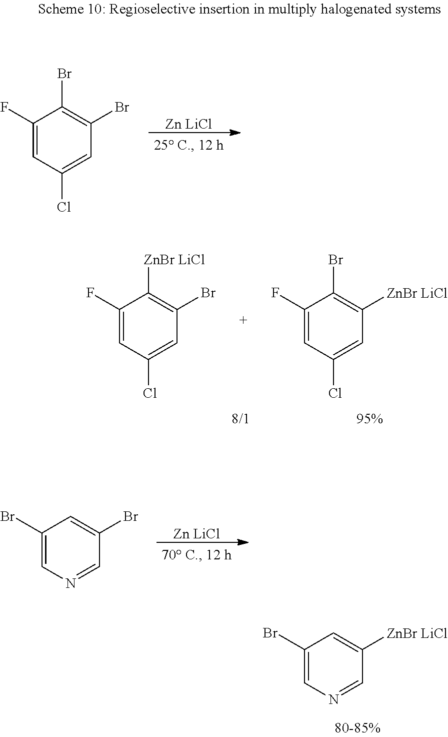

A number of di-zinc organo compounds can be prepared by the insertion of Zn in the presence of Li ions. Thereby, zinc is inserted in several iodine-carbon bonds such as illustrated in the examples in scheme 11. On the other hand, it is also possible to prepare di- or tri-organo element compounds with multivalent metals such as, for example, zinc. As shown in the third example of scheme 11, a dibromine compound can react with a single metal, for example, zinc. For example, the cyclic zinc pentane-1,5-diyl thus results from linear 1,5-dibromopentane which can be further reacted with an electrophile such as, for example, acetylchloride (AcCl). Thereby, two arms of the linear pentane are coordinated at a single zinc atom. From this example, there can be seen that also several mono-halogen compounds can be reacted with a single metal to di- or tri-organo element compounds.

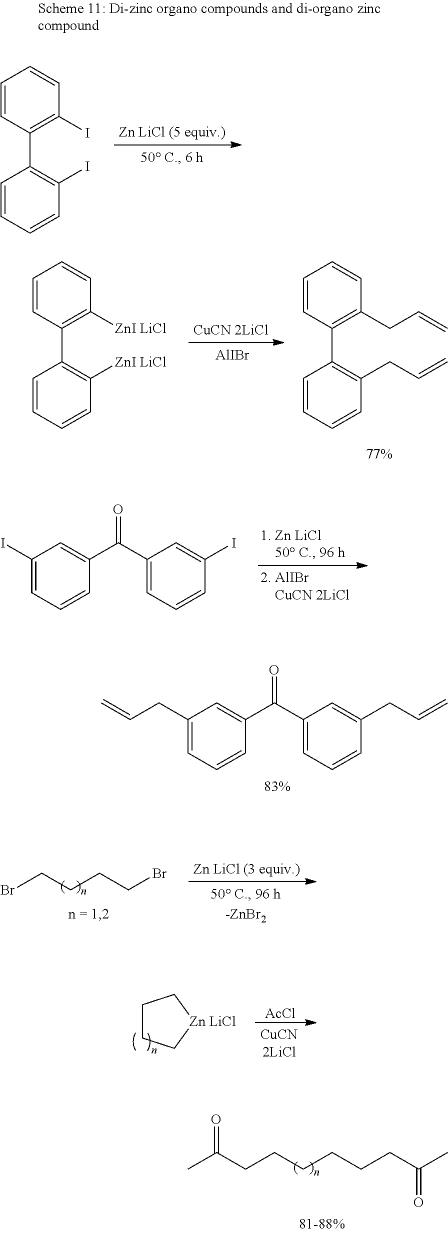

According to another embodiment of the present invention, the insertion reaction can be accelerated by the addition of amines. Thus, compounds which could originally not be reacted under conventional reaction conditions can now be made accessible to a reaction procedure according to the invention. In a preferred further embodiment, the insertion of zinc is accelerated by the addition of amines.

Any amines known to a person skilled in the art can be used as amines. These include primary, secondary and tertiary amines. Oligo- and polyamines are most preferably used. Most preferred amines are shown in scheme 12 below.

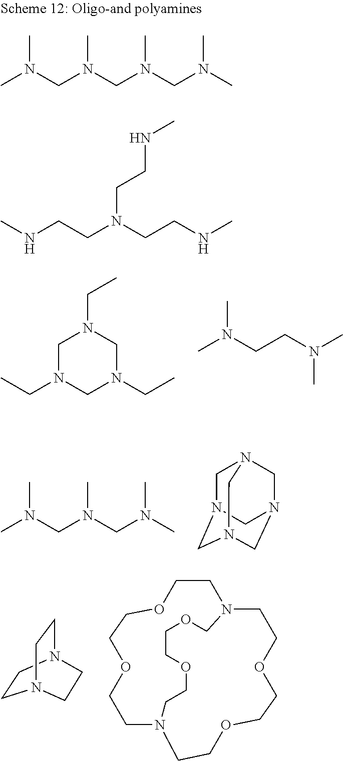

The amines can be added in any amount. Preferably, the amines are added in an amount of from 0.05 to 3 equivalents, more preferably in an amount of from 0.15 to 1.5 equivalents and most preferably from 0.2 to 1 equivalents in relation to the amount of the element M¹ and/or the metal M², specifically zinc, that is added.

In table 1 below, there are presented different reagents which have been reacted according to the general synthesis instruction for 3a. Thereby, a good yield is shown after adding N,N,N′,N′N″-pentamethyl diethylene triamine as the amine (“amines”). The addition of CuCN was carried out in order to react the zinc species into a more reactive Cu species catalytically.

TABLE 1
Preparation and reaction of aryl- and heteroaryl-functionalised zinc reagents in the
presence of N,N,N′,N′N″-pentamethyl diethylene triamine (“amines”).

| | | temperature | time | | |
|---|---|---|---|---|---|
| No. | zinc reagent, yield (%)^{[a]} | [° C.] | [h] | electrophile | product, yield (%)^{[b]} |
| 1 | 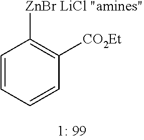 | 50 | 10 | t-BuCOCl^{[b]} | 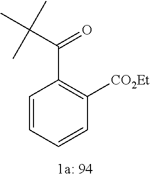 |
| 2 | 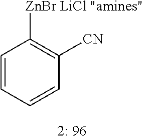 | 50 | 24 | AllBr^{[a]} | 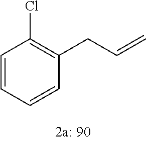 |
| 3 | 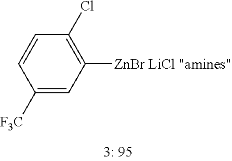 | 50 | 15 | 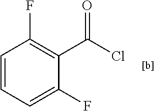 | 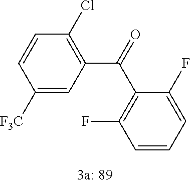 |
| 4 | 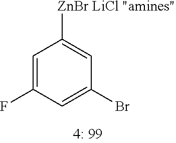 | 50 | 12 | 4- BrPhCOCl^{[b]} | 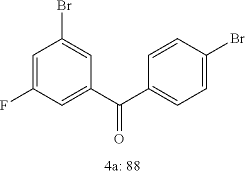 |
| 5 | 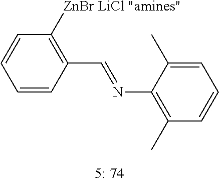 | 50 | 76 | AllBr^{[a]} | 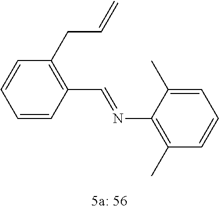 |
| 6 | 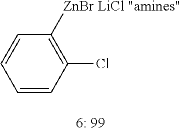 | 50 | 170 | PhCOCl^{[b]} | 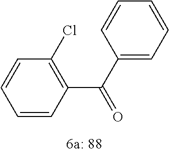 |
| 7 | 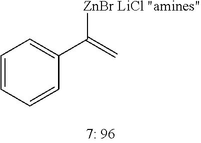 | 50 | 3.5 | AllBr^{[a]} | 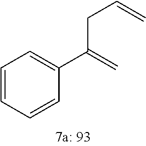 |
| 8 | 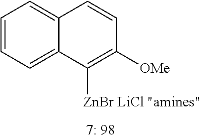 | 50 | 48 | 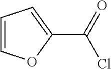 | 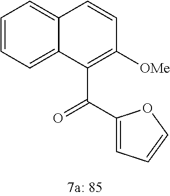 |
| 9 | 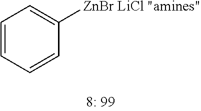 | 50 | 1 | AllBr^{[a]} | 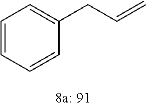 |
| 10 | 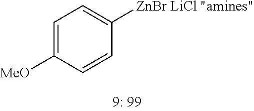 | 50 | 3 | AllBr^{[a]} | 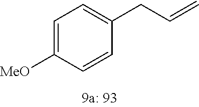 |

^{[a]}2 mol % CuCN 2LiCl has been added.
^{[b]}30 mol % CuCN 2LiCl has been added.
(Bu = Butyl, All = Allyl, Ph = Phenyl)

Hereinafter, the reaction procedure shall be illustrated by use of typical synthesis instructions. These instructions shall serve as exemplary reaction procedures and can be modified by a person skilled in art in accordance with his expertise for preparing other reaction products. The reactions shall not limit the invention in any way.

### Typical Synthesis Instructions

### Preparation of 4-ethoxy-4-oxobutyl zinc bromide

In a 25 ml-Schlenk flask, LiCl (636 mg, 15 mmol) is provided and dried with a hot air blower at 140° C. under high vacuum for 10 min. Zinc powder (981 mg, 15 mmol) as well as dry THF (12 ml) and 1,2-dibromomethane (20 μl, 0.225 mmol) are provided in a flask and carefully heated to 60° C. for 1 min. under argon. After cooling to 35° C., Me₃SiCl (20 μl, 0.102 mmol) is added and vigorously stirred for 15 min. The reaction is tempered to 50° C. in an oil bath and 4-bromobutane acid ethylester (975 mg, 5 mmol) is slowly added through a septum. The reaction control is carried out by the use of a GC. After 1 h, no educt is detected any more.

### Preparation of [4-(ethoxycarbonyl)phenyl] zinc bromide

In a 25 ml-Schlenk flask, LiCl (636 mg, 15 mmol) is provided and dried with a hot air blower at 140° C. under high vacuum for 10 min. Zinc powder (981 mg, 15 mmol) as well as dry THF (12 ml) and 1,2-dibromomethane (20 μl, 0.225 mmol) are provided in a flask and carefully heated to 60° C. for 1 min. under argon. After cooling to 35° C., Me₃SiCl (20 μl, 0.102 mmol) is added and vigorously stirred for 15 min. The reaction is tempered to 50° C. in an oil bath and 4-bromo benzoic acid ethylester (1145 mg, 5 mmol) is slowly added through a septum. The reaction control is carried out by the use of a GC. After 18 h, no educt is detected any more.

### Preparation of [2-chloro-5-(trifluoromethyl)phenyl]-(2,6-difluorophenyl)methanone (3a)

Anhydrous LiCl (16 mol) is introduced in a 25 ml-Schlenk flask having been rinsed with argon and dried under high vacuum (<1 mbar) at 150-170° C. for 5 minutes. Zinc powder (15 mmol) is added under argon and the flask is three-times evacuated and filled with argon. Then, dry THF (10 ml) is added and the zinc is activated with BrCH₂CH₂Br (5 mol %) and Me₃SiCl (1 mol %). The mixture is heated to 50° C. and then, 2-bromo-1-chloro-4-(trifluoromethyl)benzene (5 mmol) in 2 ml dry THF with an internal standard (n-tetradecane) of about 10% are added, followed by 5 mmol N,N,N′,N′,N″-pentamethyldiethylenetetramine. The insertion reaction is completed after 15 hours (control by use of an GC analysis of reaction aliquots wherein the reaction has proceeded for more than 99%). The solution of bromo-[2-chloro-5-(trifluoromethyl)phenyl] zinc (2.5 mmol, 5.5 ml) is carefully separated from the remaining zinc powder by use of a syringe and transferred into another 10 ml-Schlenk flask having been rinsed with argon. CuCN2LiCl (0.75 ml of a 1.0 M solution in THF, 0.75 mmol, 30 mol %) is added at −20° C., followed by 2,6-difluorobenzoylchloride (3.5 mmol). The reaction mixture is stirred over 1 hour at 0° C. and then quenched with a saturated aequeous solution of NH₄Cl (5 ml). The aequeous phase is extracted with EtOAc (3×5 ml) and concentrated in vacuo. The raw product is purified via flash chromatography (PE: diethylether) whereby [2-chloro-5-(trifluoromethyl)phenyl]-(2,6-difluorophenyl)methanone (3a; 1.95 mmol, 625 mg, 78%) can be obtained as white needles.

While the invention has been described with the use of concrete embodiments hereinabove, it should not be limited thereto. It is apparent for a person skilled in the art that the above examples can be modified in many ways without departing from the scope of protection of the claims. Thus, it is, for example, possible to multiply modify the reaction temperatures or times as well as the solvents or reagents. The scope of protection shall thus solely be defined by the claims.

What is claimed is:

1. Method for preparing a compound having the general formula
R¹-M¹-A_{d}zLiX (I)
by reacting a compound R¹-A (III) with an element M¹ in presence of LiX, wherein
R¹ is a substituted or un-substituted C₃-C₂₄ aryl or C₃-C₂₄ heteroaryl containing one or more heteroatoms like B, O, N, S, Se, P or Si, a linear or branched substituted or un-substituted C₁-C₂₀ alkyl, C₂-C₂₀ alkenyl or C₂-C₂₀ alkynyl or a substituted or un-substituted C₃-C₂₀ cycloalkyl or a derivative thereof;
M¹ is an element selected from Mn, Cu, Zn, Sn, In, La, Ce, Nd, Y, Li, Sm, Na, K and Bi;
A is a halogen selected from F, Cl, Br, I; or a sulphonate (RSO₃—) or a phosphonate (—OP(O)(OR)₂) wherein R is defined as R¹,
d is 0 or 1;
z is >0; and
X is selected from the group consisting of F; Cl; Br; CN; SCN; NCO; Hal¹Oₖ,
wherein k=3 or 4 and Hal¹ is selected from Cl, Br and I; NO₃; BF₄; PF₆; H; a carboxylate having the general formula R^{x}CO₂; a disilazide having the general formula (R^{x}₃Si)₂; a thiolate having the general formula SR^{x}; an alcoholate having the general formula OR^{x}; R^{x}P(O)O₂; or SCOR^{x}; an amine having the general formula R^{x}NH; a dialkyl- or diarylamine having the general formula R^{x}₂N, wherein R^{x} is defined as below or R^{x}₂N represents a cyclic alkylamine; a phosphine having the general formula PR^{x}₂, wherein Rx is defined as below or PR^{x}₂ represents a cyclic phosphine; OⱼSR^{x}, wherein j=2 or 3; or NOᵣ, wherein r=2 or 3; and derivatives thereof; wherein R^{x} is a substituted or un-substituted C₄-C₂₄ aryl or a C₃-C₂₄ heteroaryl containing one or more heteroatoms like B, O, N, S, Se, P or Si; a linear or branched substituted or un-substituted C₁-C₂₀ alkyl; C₂-C₂₀ alkenyl or C₂-C₂₀ alkynyl; or a substituted or un-substituted C₃-C₂₀ cycloalkyl; or derivatives thereof; or H.

2. Method for preparing a compound having the general formula
R¹ₘ-M³-TₙzLiX (II)
by reacting a compound R¹-A (III) with a M³-containing compound in the presence of LiX and in the presence of an elementary metal M² wherein
R¹, z, A and X are defined as in claim 1;
T is defined as A or X in claim 1 and wherein X and T can be identical or different;
M³ is defined as M¹ in claim 1 and additionally comprises Ti, Al, Mg, B, Si and S;
n is 0, 1, 2 or 3;
m is 1, 2 or 3;
M² is a metal being selected from Li, Na, K, Cs, Mg, Ca, Mn and Zn and the moieties R¹ can be identical or different, when m=2 or m=3.

3. Method according to claim 2, wherein the M³-containing compound is selected from metal-halogen compounds, metal-alkyl compounds, metal-aryl compounds, metal-alkoxy compounds or metal-aryloxy compounds.

4. Method according to claim 2 or 3 wherein the M³-containing compound is selected from MgBr₂, MgCl₂, B(OMe)₃, B(iPrO)₃, BF₃, Et₂AlCl, Si(OMe)₄, SiCl₄, MnCl₂, SnCl₂, ZnCl₂, ZnBr₂, TiCl(OiPr)₃, Ti(OiPr)₄, InCl₃, LaCl₃, CeCl₃, SmCl₃ and NdCl₃.

5. Method according to claim 1 or 2, wherein the method is carried out in a solvent selected from cyclic, linear or branched mono- or polyethers, thioethers, amines, phosphines and derivatives thereof that contain one or more additional heteroatoms selected from O, N, S and P, preferably tetrahydrofurane (THF), 2-methyltetrahydrofurane, dibutylether, diethylether, tert-butylmethylether, dimethoxyethane, dioxanes, preferably 1,4-dioxane, triethylamine, ethyldiisopropylamine, dimethylsulfide, dibutylsulphide; cyclic and linear amides, preferably N-methyl-2-pyrrolidone (NMP), N-ethyl-2-pyrrolidone (NEP), N-butyl-2-pyrrolidone (NBP), N,N-dimethylformamide (DMF), N,N-dimethylacetamide (DMAC); cyclic, linear or branched alkanes and/or alkenes wherein one or more hydrogen atoms are replaced by halogens, preferably dichlormethane, 1,2-dichlorethane, CCl₄; derivatives of urea, preferably N,N′-dimethylpropylene urea (DMPU), N,N,N′N′-tetramethyl urea; aromatic, heteroaromatic or aliphatic hydrocarbons, preferably benzene, toluene, xylene, pyridine, pentane, cyclohexane, hexane, heptane; hexamethylphosphorotriamide (HMPA), CS₂; or combinations thereof.

6. Method according to claim 1 or 2, characterised in that the elementary metal M¹ or M² is activated with a compound selected from the group consisting of copper salts, nickel salts, iron compounds, cobalt compounds, I₂, C₂H₄Br₂, Cl(CH₂)₂Br, t-BuOLi, BCl₃, BF₃, LiBH₄, LiAlH₄, NaAlH₄, Et₃Al, DIBAL-H, Na[H₂Al(OCH₂CH₂OCH₃)]Me₃SiCl, Et₂Zn, ICl and SnCl₂.

7. Method according to claim 1 or 2, characterised in that M¹ or M² is Zn.

8. Method according to claim 2, characterised in that, when n 2, T₂ is a bivalent anion selected from the group consisting of diamines, dialkoxides or dithiols.

9. Method according to claim 8 characterised in that the diamine has the general formula R′NH—R—NHR′, the dialkoxide has the general formula HO—R—OH and the dithiol has the general formula HS—R—SH, wherein R′ and R are independently from each other selected from the same group as R^{x}, wherein R is a bivalent moiety and preferably CH₃NHCH₂CH₂NHCH₃, HOCH₂CH₂OH, binole, 1,2-diaminocyclohexane are used.

10. Method according to claim 1 or 2, characterised in that an amine, preferably an oligo- or polyamine, is added additionally.

11. Method according to claim 10 characterised in that the amine is added in an amount of from 0.05 to 3 equivalents, preferably from 0.15 to 1.5 equivalents, more preferably from 0.2 to 1 equivalents, in relation to the element M¹ and/or the metal M².

12. Compound having the general formula
R¹ₘ-M³-TₙzLiX (II)
wherein R¹, z and X are defined as in claim 1 and n, m, T and M³ are defined as in claim 2, but M³ does not comprise Mg.

13. Solution of a compound having the general formula
R¹ₘ-M³-TₙzLiX (II)
wherein R¹, z and X are defined as in claim 1 and n, m, T and M³ are defined as in claim 2, but M³ does not comprise Mg in a solvent.

14. Solution according to claim 13 wherein the method is carried out in a solvent selected from cyclic, linear or branched mono- or polyethers, thioethers, amines, phosphines and derivatives thereof that contain one or more additional heteroatoms selected from O, N, S and P, preferably tetrahydrofurane (THF), 2-methyltetrahydrofurane, dibutylether, diethylether, tert-butylmethylether, dimethoxyethane, dioxanes, preferably 1,4-dioxane, triethylamine, ethyldiisopropylamine, dimethylsulfide, dibutylsulphide; cyclic and linear amides, preferably N-methyl-2-pyrrolidone (NMP), N-ethyl-2-pyrrolidone (NEP), N-butyl-2-pyrrolidone (NBP), N,N-dimethylformamide (DMF), N,N-dimethylacetamide (DMAC); cyclic, linear and branched alkanes and/or alkenes wherein one or more hydrogen atoms are replaced by halogens, preferably dichlormethane, 1,2-dichlorethane, CCl₄; derivatives of urea, preferably N,N′-dimethylpropylene urea (DMPU), N,N,N′N′-tetramethyl urea; aromatic, heteroaromatic or aliphatic hydrocarbons, preferably benzene, toluene, xylene, pyridine, pentane, cyclohexane, hexane, heptane; hexamethylphosphorotriamide (HMPA), CS₂; or combinations thereof.

15. Use of a compound having the general formula
R¹ₘ-M³-TₙzLiX (II)
wherein R¹, z and X are defined as in claim 1 and n, m, T and M³ are defined as in claim 2, but M³ does not comprise Mg in a reaction with an electrophile.

16. Product of a reaction of an electrophile with a compound having the general formula
R¹ₘ-M³-TₙzLiX (II)
wherein R¹, z and X are defined as in claim 1 and n, m, T and M³ are defined as in claim 2, but M³ does not comprise Mg.
